(19) United States
(12) Patent Application Publication
(10) Pub. No.: US 2005/0164969 A1
(43) Pub. Date: Jul. 28, 2005

(54) Method of extending life span

(75) Inventors: Gil Blander, Cambridge, MA (US); Leonard Guarente, Brookline, MA (US)

Correspondence Address: PROTEUS PATENT PRACTICE LLC, P.O. BOX 1867, NEW HAVEN, CT, 06508, US

(73) Assignee: Massachusetts Institute of Technology, Cambridge, MA (US)

(21) Appl. No.: 10/993,903

(22) Filed: Nov. 19, 2004

Related U.S. Application Data
(60) Provisional application No. 60/481,665, filed on Nov. 19, 2003.

(51) Int. Cl. A61K 48/00; C07H 21/04

(52) U.S. Cl. 514/44; 435/455; 530/350; 536/23.5

(57) ABSTRACT

The present invention provides new and advantageous methods, compositions, cell constructs and animal models related to inhibiting the senescence of vertebrate cells and vertebrate organisms based on the use of SIRT1 polynucleotides and polypeptides, as well as mutant SIRT1 polynucleotides and polypeptides. The invention provides polynucleotides that encode variants and fragments of SIRT1 polypeptides, and also provides variant SIRT1 polypeptides and fragments thereof. Additionally the invention provides a method of inhibiting or delaying the expression in a vertebrate cell of a protein having biological activity associated with loss of population doubling in the cell. The invention further provides a method of treating a pathology, a disease or a medical condition in a subject, wherein the pathology responds to an SIRT1 polypeptide. The invention also provides a vertebrate cell that incorporates a heterologous nucleic acid encoding a variant of SIRT1, or a fragment thereof, as well as a transgenic mammal a majority of whose cells harbor a transgene including a nucleic acid sequence encoding an SIRT1 polypeptide. The invention also provides an antibody that binds immunospecifically to a variant SIRT1 polypeptide or a fragment thereof, and a method of determining whether the amount of an SIRT1 polypeptide in a sample differs from the amount of the SIRT1 polypeptide in a reference. The invention further provides a method of contributing to the diagnosis or prognosis of, or to developing a therapeutic strategy for, a disease or pathology in a subject, wherein the disease or pathology responds to treatment with an SIRT1 polypeptide and wherein the amount of SIRT1 polypeptide in the pathology is known to differ from the amount of the SIRT1 polypeptide in a nonpathological state.

### RELATED APPLICATIONS

This application claims the benefit of priority of U.S. Provisional Application Ser. No. 60/481,665 filed Nov. 19, 2003.

### STATEMENT REGARDING FEDERAL FUNDING

The present invention was made with Government support and the Government has certain rights in the invention.

### REFERENCE TO A SEQUENCE LISTING

This application includes one (1) Compact Disc containing a Sequence Listing. The file name containing the Sequence Listing is 407-01B SEQLIST.

### BACKGROUND OF THE INVENTION

SIRT1

The human ortholog of yeast SIRT2 (silent mating type information regulation 2), SIRT1, is an NAD⁺-dependent deacetylase (Imai S et al. Cold Spring Harb Symp Quant Biol. 2000; 65: 297-302). The SIRT1 protein is localized in the nucleus (Luo J et al. Cell. 2001; 107(2): 137-48; Vaziri H et al. Cell. 2001; 107(2): 149-59). SIRT1 interacts with and deacetylates a large number of proteins. A knockout mouse showed that this protein is important for embryonic development. The protein has also been shown to play a role in muscle differentiation. Moreover, SIRT1 appears to increase expression of hTERT when overexpressed (Lin S Y et al. Cell 2003; 113(7): 881-9), suggesting that it may function as an hTERT activator.

Protein Substrate and Protein Interaction

Several protein-protein interactions involving SIRT1 have been identified. Following DNA damage, the p53 protein is acetylated, resulting in activation. In view of the deacetylase activity of SIRT1 interaction between SIRT1 and p53 was investigated. Vaziri et al. (2001) and Luo et al. (2001) showed independently that p53 and SIRT1 co-immunoprecipitate each other in transiently transfected cells and from endogenous proteins. DNA-damaging agents augment in vivo interaction (Luo et al., 2001). In vitro an NAD⁺ dependent deacetylation of a p53 peptide including acetylated lysine 382 has been observed (Luo et al., 2001; Langley E et al. EMBO J. 2002; 21(10): 2383-96). Upon exposure of immortalized human fibroblast to ionizing radiation, a marked increase in the p53 acetylation level was detected. The increase in the acetylation levels was abrogated in cells that overexpress the SIRT1 protein (Vaziri et al., 2001). Deacetylation of p53 leads to apoptosis (Vaziri et al., 2001; Luo et al., 2001; Langley et al., 2002). A modified SIRT1 carrying a point mutation in the deacetylase motif functions as a dominant negative mutant by inhibiting p53 deacetylation, promoting p53-dependent apoptosis (Vaziri et al. 2001).

The nuclear bodies (NB), often termed promyelocytic leukemia protein (PML) NB, are distinct nuclear substructures that accumulate PML proteins (Seeler J S et al. Curr Opin Genet Dev. 1999; 9(3): 362-7). It has been found that endogenous SIRT1 interacts with PML4. When SIRT1 was co-expressed with PML4, it was localized to the PML-NB (Langley et al., 2002). Moreover, SIRT1 and PML4 co-localized with p53 in the PML-NB. Over expression of PML4 in primary cells leads to immediate growth arrest. Interestingly, SIRT1 co-expression rescued the cells from the growth arrest (Langley et al., 2002). Together, these results indicate that SIRT1 may be a positive effector of cell growth that negatively regulates p53 and PML.

CTIP2 is a sequence-specific, DNA binding protein that represses transcription via direct DNA binding. SIRT1 binds to CTIP2 in vivo and in vitro, and is recruited to CTIP2 target promoter sequences in a CTIP2-dependent manner. SIRT1 stimulates the repression by CTIP2 and enhances the histone deacetylation of a CTIP2 target promoter (Senawong T et al. J Biol. Chem. 2003; 278(44): 43041-50. Epub 2003 Aug. 19). These data suggest that SIRT1 can be recruited to promoters by specific transcription factors, and functions to repress the transcription of specific genes.

The expression of muscle cell genes is regulated by acetylation and deacetylation (Sartorelli V et al. Front Biosci. 2001; 6: D1024-47). The Sartorelli group showed that mouse SIRT1 negatively regulates skeletal muscle differentiation. SIRT1 overexpression negatively regulates the transcription of those genes and prevents full differentiation into muscle cells. The PCAF protein mediates the interaction between SIRT1 and the transcription factor MyoD. In vitro SIRT1 deacetylates MyoD and PCAF in an NAD⁺-dependent manner. Many genes that are activated by MyoD and involved in myogenesis are repressed by SIRT1. In addition it was found that SIRT1 is recruited to the MyoD targets and deacetylates histones in the target promoters. (Fulco M et al. Mol Cell. 2003; 12(1): 51-62.).

SIRT1 Knockout Mice

In SIRT1 knockout mice, the proportion of homozygous knockout mice was lower than was expected. The lower proportion of the null animals at birth reflects the immediate postnatal loss of abnormal fetuses. The mice are smaller than their wild type littermates and most of them die during the first few months after birth (McBurney M W et al. Mol Cell Biol. 2003; 23(1): 38-54; Cheng H L et al. Proc Natl Acad Sci USA. 2003; 100-(19): 10794-9; Epub 2003 Sep. 05). In addition several developmental defects are noted in the knockouts. The p53 acetylation level is much higher in the knockout mice.

The present inventors have identified several novel compositions, cell constructs and methods related to SIRT1 for which there is an unmet need. For example, there is a need for extending the life span of a cell and/or its progeny, and for inhibiting or retarding differentiation, among others. These needs are addressed herein.

### SUMMARY OF THE INVENTION

The present invention provides new and advantageous methods, compositions, cell constructs and animal models related to inhibiting the senescence of vertebrate cells and vertebrate organisms based on the use of SIRT1 polynucleotides and polypeptides, as well as mutant SIRT1 polynucleotides and polypeptides.

In a first aspect, the invention provides an isolated polynucleotide that includes a nucleotide sequence chosen from among:
a) a nucleotide sequence encoding a variant SIRT1 polypeptide whose amino acid sequence is at least 90% identical to an amino acid sequence that differs from the sequence given by SEQ ID NO:1 by one amino acid residue;
b) a nucleotide sequence complementary to a nucleotide sequence described in a);
c) a nucleotide sequence that is a fragment of any of the nucleotide sequences of a) or b); and
d) a nucleotide sequence that hybridizes to a nucleotide sequence given by a) through c).

In a second aspect, the invention provides an isolated variant SIRT1 polypeptide that includes a sequence chosen from among:
a) a polypeptide whose amino acid sequence is at least 90% identical to an amino acid sequence that differs from the sequence given by SEQ ID NO:1 by one amino acid residue; and
b) an amino acid sequence that is a fragment of the amino acid sequence given in a).

In both the variant polynucleotide and the variant polypeptide the encoded polypeptide exhibits at least one biological activity of SIRT1.

In a further aspect the invention provides a method of extending the population doubling of a vertebrate cell. This method includes the step of contacting the cell with a nucleic acid that includes a sequence encoding an SIRT1 polypeptide.

In still an additional aspect, the invention provides a method of inhibiting or delaying the expression in a vertebrate cell of a protein having biological activity associated with loss of population doubling in the cell. This method includes the step of contacting the cell with a nucleic acid that includes a sequence encoding an SIRT1 polypeptide. In a significant embodiment of this method, the inhibited protein is a polypeptide having beta-galactosidase activity. In an additional significant embodiment of the method of inhibiting or delaying, the method is effective to inhibit or delay a differentiation process in the cell.

In various significant embodiments of the methods described in the preceding paragraphs, the cell is a mammalian cell; and in still more significant embodiments the cell is a human cell. In still other significant embodiments of these methods, the cell is in vitro, ex vivo, or in vivo. In certain significant embodiments the cell may be a cardiac myocyte, a neuron, a glial cell, a kidney cell, an endothelial cell, a myoblast, a muscle cell, an osteoblast, an osteoclast, a fibroblast, a keratinocyte, or a dermal, epidermal, or mucosal epithelial cell.

In a further aspect the invention provides a method of treating a pathology, a disease or a medical condition in a subject, wherein the pathology responds to an SIRT1 polypeptide, the method including the step of administering a nucleic acid encoding an SIRT1 polypeptide to the subject in an amount effective to attenuate or ameliorate the pathology. In important implementations of this method the pathology, disease or medical condition is chosen from among myocardial infarction, cerebrovascular stroke, a kidney disease, a neurological disease, a traumatic wound, a surgical wound, a fractured bone, a bone having a surgical wound, a condition of a dermal, epidermal, or mucosal epithelial surface, and the like. In advantageous embodiments of the method of treating a pathology the subject is a human.

In still an additional aspect the present invention provides a vertebrate cell that incorporates a heterologous nucleic acid that includes a nucleotide sequence encoding a variant of SIRT1, or a sequence encoding a fragment of the polypeptide of SEQ ID NO:1. In an important embodiment of the vertebrate cell, the population doubling of the cell is extended with respect to the population doubling of a cell not so transfected. In another important embodiment of the vertebrate cell the mutant or variant SIRT1 polypeptide possesses a biological function of wild type SIRT1. In still other important embodiments of the vertebrate cell, the cell is in vitro, ex vivo, or in vivo. In certain important embodiments the cell may be a cardiac myocyte, a neuron, a glial cell, a kidney cell, an endothelial cell, a myoblast, a muscle cell, an osteoblast, an osteoclast, a fibroblast, a keratinocyte, or a dermal, epidermal, or mucosal epithelial cell. In a further important embodiment of the vertebrate cell, the expression in the vertebrate cell of a protein having biological activity associated with loss of population doubling in the cell is inhibited or delayed. In yet additional significant embodiments a differentiation process in the cell is inhibited or delayed.

In still an additional aspect the present invention provides a transgenic mammal a majority of whose cells harbor a transgene including a nucleic acid sequence encoding a variant of SIRT1, or a sequence encoding SIRT1 or a fragment thereof. In an advantageous embodiment of the transgenic mammal, the number of the transgenes in the majority of its cells is higher than the number of SIRT1 sequences in the cells of a nontransgenic mammal of the same species. In advantageous embodiments, the life span of those cells in the transgenic mammal that express an SIRT1 polypeptide is increased with respect to a nontransgenic mammal of the same species. In an additional advantageous embodiment, the heterologous nucleic acid further includes one or more of an enhancer sequence, a promoter sequence, and a polyadenylation sequence each of which is operably linked to the SIRT1 sequence.

In yet a further aspect the invention discloses an antibody that binds immunospecifically to a variant SIRT1 polypeptide or a fragment thereof.

In still an additional aspect the invention provides a method of determining whether the amount of an SIRT1 polypeptide in a sample differs from the amount of the SIRT1 polypeptide in a reference. This method includes the steps of:
a) providing a sample suspected to include the SIRT1 polypeptide;
b) contacting the sample with a specific binding agent that binds an SIRT1 polypeptide under conditions that assure binding of the SIRT1 polypeptide to the specific binding agent; and
c) determining whether the amount of the specific binding agent that binds to the sample differs from the amount of the specific binding agent that binds to a reference under the same conditions used in step b), wherein the reference comprises a standard or reference amount of the SIRT1 polypeptide.

In yet an additional aspect the invention provides a method of contributing to the diagnosis or prognosis of, or to developing a therapeutic strategy for, a disease or pathology in a first subject, wherein the disease or pathology responds to treatment with an SIRT1 polypeptide and wherein the amount of SIRT1 polypeptide in the pathology is known to differ from the amount of the SIRT1 polypeptide in a nonpathological state. This method includes the steps of:
a) providing a sample from the first subject suspected to include the SIRT1 polypeptide;
b) contacting the sample with a specific binding agent that binds an SIRT1 polypeptide under conditions that assure binding of the SIRT1 polypeptide to the specific binding agent; and
c) determining whether the amount of the specific binding agent that binds to the sample differs from the amount of the specific binding agent that binds to a reference under the same conditions used in step b), wherein the reference is provided from a second subject known not to have the pathology;
thus contributing to the diagnosis or prognosis of, or to developing a therapeutic strategy for, the pathology.

In significant embodiments of the methods described in the preceding two paragraphs, the specific binding agent is an antibody.

### BRIEF DESCRIPTION OF THE FIGURES

FIG. 1. Western blot of SIRT1 in WI-38 cells at PDL 33 and 49.

FIG. 2. Western blot of SIRT1 and related proteins expressed in WI-38 cells under various experimental conditions.

FIG. 3. Graphical representation of the PDL attained by WI-38 cells at various times after plating of transfected cells. The ordinate shows PDL.

FIG. 4. Graphical representation of beta galactosidase activity in transfected WI-38 cells at 31 days.

FIG. 5. Western blot of SIRT1 and related proteins expressed in MRC-5 cells under various experimental conditions.

FIG. 6. Graphical representation of the PDL attained by MRC-5 cells at various times after plating of transfected cells.

FIG. 7. Photomicrograph of MRC-5 cells on day 57 stained with X-gal for beta-galactosidase activity.

FIG. 8. Graphical representation of beta galactosidase activity in transfected MRC-5 cells on day 57.

### DETAILED DESCRIPTION OF THE INVENTION

The present disclosure includes a Sequence Listing. A correspondence of the sequences is provided in Table 1.

TABLE 1
Sequence Listing Correspondence

| | GenBank | |
|---|---|---|
| Description | Accession No. | SEQ ID NO: |
| Human SIRT1 protein | NP 036370 | 1 |
| Human SIRT1 mRNA | NM_012238 | 2 |
| 5′ primer-1 (PCR primer) | | 3 |
| 3′ primer-2 (PCR primer) | | 4 |
| Primer-3 (PCR primer) | | 5 |

As used herein, the terms “population doubling” and “population doubling number” (both of which are abbreviated PDL) relate to the number of times a parental cell has divided to produce progeny cells. Generally each cell division produces two progeny cells. In the context of the present invention it is recognized in the field that, at the time the present invention was made, there was a limit recognized in fields related to the invention, termed the “Hayflick limit”, to the PDL value for normal vertebrate cells.

As used herein the term “transfected” and similar terms and phrases relate to a vertebrate cell in culture into which a heterologous nucleic acid, or gene or fragment thereof, or a plasmid or vector containing such a heterologous sequence, has been introduced. Transfection may be transient or may result in permanent incorporation of the heterologous nucleic acid. A “heterologous” nucleic acid, gene or fragment thereof is any such construct that is not a component of the wild type cell.

As used herein the term “transformed” and similar terms and phrases relate to a vertebrate cell into which a heterologous nucleic acid, or gene or fragment thereof, or a plasmid or vector containing such a heterologous sequence, has been introduced. Transformation results in a permanent or heritable incorporation of the heterologous nucleic acid. A “heterologous” nucleic acid, gene or fragment thereof is any such construct that is not a component of the wild type cell.

As used herein “attenuating”, and similar terms and phrases, when considering symptoms of a disease or pathology, signifies that a trend of worsening symptomology is abated to a slower or more gentle trend of worsening. As used herein “ameliorating”, and similar terms and phrases, when considering symptoms of a disease or pathology, signifies an actual improvement in a subject, such that the signs and indications of disease diminish, and the subject improves toward better health.

The present invention relates to several aspects in which a gene product of a nucleic acid encoding an SIRT1 polypeptide acts within a vertebrate cell, or within a vertebrate organism, to inhibit senescence and/or to extend population doubling. Generally as used herein “inhibiting senescence” and “extending population doubling”, and similar terms and phrases, relate to carrying a cell up to and beyond a cell's Hayflick limit, and to retarding cellular processes associated with approach to the Hayflick limit. In a first aspect, the invention discloses introducing a nucleic acid containing a sequence encoding an SIRT1 polypeptide into a vertebrate cell effective to retard the onset of senescence, to promote the extension of the population doubling number, and/or to inhibit a differentiation process of the cell. The vertebrate cell so transformed may be in an in vitro cell culture, or it may be in an ex vivo tissue or organ sample, or it may exist in vivo as a constituent of a living organism. In many significant exemplifications of the invention the transfected or transformed cell is a mammalian cell; and still more significantly the cell is a human cell.

In all the various methods described herein, the nucleic acid encoding the SIRT1 polypeptide may be a naked DNA molecule, or it may be a component of a plasmid, a cosmid, a phagemid, an artificial chromosome, a virus particle or virus-like particle, a liposome, or any similar or equivalent vector which effectively acts to introduce the SIRT1 nucleotide sequence into the cell. Furthermore the SIRT1 nucleic acid advantageously is operably linked to at least one element such as an enhancer, a promoter, or a polyadenylation site that serve to promote the de novo intracellular expression of the encoded SIRT1 polypeptide.

In an additional aspect, the present invention discloses a method of inhibiting or delaying the expression in a vertebrate cell of a protein having biological activity associated with cessation of population doubling in the cell. Many effects related to senescence involve preferential increase in an enzymatic activity or in a ligand-binding pathway, such as a signaling pathway. An important implementation of the present invention includes inhibiting, retarding, or minimizing such biological function or activity. Although many such activities are known or are inherent in a cell, a nonlimiting example of such an activity is ascribed to a polypeptide having beta-galactosidase activity. The transfected or transformed vertebrate cell may be in an in vitro cell culture, or it may be in an ex vivo tissue or organ sample, or it may exist in vivo as a constituent of a living organism. In many significant exemplifications of the invention the cell is a mammalian cell; and still more significantly the cell is a human cell. Importantly, when introduced into several types of vertebrate cell and expressed therein, an SIRT1 polynucleotide of the invention induces an inhibition or a delay of a differentiation process of the cell.

As used herein the term “differentiation” and similar terms relate to a process in which a cell progresses from a state that is relatively nonspecialized to one that is more particularly specialized. Specialization of a cell may be characterized by morphology, ultrastructural features, nucleic acid or polypeptide expression profiles, activities, and the like. As used herein “differentiation” includes a process leading to necrotic cell death or to apoptotic cell death.

In the several embodiments of the methods described in the preceding paragraphs, the nucleic acid encoding an SIRT1 polypeptide may be chosen from among a variety of constructs that ensure efficient delivery of the nucleic acid sequence into cells, including into cells of a subject. These constructs include, by way of nonlimiting example, a naked DNA molecule; a plasmid or similar vector; a virus or virus-like particle, such as an engineered retrovirus, an engineered adenovirus or an adeno-associated virus, whose nucleic acid includes an SIRT1 sequence; a vesicle that includes a polynucleotide encoding an SIRT1 sequence; and similar effective compositions. All the constructs transfect or transform the target cells by introducing an SIRT1 coding sequence into the cell in such a way as to promote the de novo expression of the encoded SIRT1 polypeptide. In many embodiments a naked DNA, a plasmid or vector, a virus or a polynucleotide of the vesicle will include one or more of an enhancer sequence, a promoter sequence, and a polyadenylation sequence each of which is operably linked to the SIRT1 sequence. These constructs enhance the efficiency of the de novo synthesis of SIRT1 within a transfected or transformed cell. Any equivalent nucleic acid that serves to introduce an SIRT1-encoding nucleic acid into a cell and that enhances de novo synthesis of an SIRT1 polypeptide falls within the scope of the invention.

In still an additional aspect the present invention provides a vertebrate cell that incorporates a heterologous nucleic acid containing a sequence encoding an SIRT1 polypeptide. Such a cell is termed a “modified vertebrate cell”, and includes a “transfected vertebrate cell” or a “transformed vertebrate cell” herein. A significant attribute of the modified vertebrate cell is that its population doubling number is extended, compared to the population doubling of a cell that has not been treated to include a heterologous SIRT1 sequence. As a consequence of such a vertebrate cell expressing a functional form of an SIRT1 polypeptide, expression in the modified vertebrate cell of a protein having biological activity associated with loss of population doubling in the cell may be inhibited or delayed. Additionally a differentiation process in the modified vertebrate cell expressing an SIRT1 polypeptide may be inhibited or delayed. In additional significant embodiments, the heterologous nucleic acid further includes one or more of an enhancer sequence, a promoter sequence, and a polyadenylation sequence each of which is operably linked to the SIRT1 sequence.

In addition, a modified vertebrate cell may be transfected or transformed with a nucleic acid sequence that encodes a mutant form of an SIRT1 polypeptide. In such mutants, one or more amino acid residues are mutated from the amino acid residue present at a given position in the wild type form of SIRT1. Such a mutant form of an SIRT1 polypeptide retains at least one biological function or activity of a wild type SIRT1 polypeptide. A full general description of an SIRT1 polypeptide, as employed in the present invention, is provided below.

The modified vertebrate cell is useful as a research tool, permitting characterization of various biological functions and activities ascribable to expression of the heterologous SIRT1 protein. Such investigations are expected to lead to additional beneficial discoveries and inventions related to promoting human health and longevity. The use of modified human cells in this way is exemplified in the Examples of this invention (see below). In addition, a modified cell of the invention may serve as a source of ex vivo cells for therapeutic use in various pathologies, diseases and medical conditions.

The present invention also provides a transgenic mammal one or more of whose cells incorporate a heterologous nucleic acid that includes a sequence encoding an SIRT1 polypeptide. In advantageous embodiments, the life span of cells in the transgenic mammal that express the heterologous SIRT1 sequence is increased with respect to a nontransgenic mammal of the same species. In an additional advantageous embodiment, the heterologous nucleic acid further includes one or more elements chosen from an enhancer sequence, a promoter sequence, and a polyadenylation sequence each of which is operably linked to the SIRT1 sequence. Such an element enhances the de novo expression of an SIRT1 polypeptide in the transgenic mammal. The transgenic mammal is useful as a research tool, permitting characterization of various biological functions and activities ascribable to expression of the heterologous SIRT1 protein. The transgenic mammal of the invention may serve as an experimental animal model for treating and ameliorating various pathologies, diseases and medical conditions. Such investigations are expected to lead to new and useful discoveries and inventions related to promoting human health and longevity.

In still further aspects the invention provides mutant SIRT1 polypeptides and polynucleotides encoding a mutant SIRT1 polypeptide, wherein the SIRT1 polypeptides retain at least one biological activity or function of wild type SIRT1.

SIRT1

As used herein, the terms an “SIRT1 polypeptide”, an “SIRT1 protein”, and related terms and phrases, relate to wild type SIRT1, to a mutant SIRT1, a variant SIRT1, and to fragments and mature forms thereof. An important SIRT1 protein to be used in the present invention is human SIRT1. The amino acid sequence of SIRT1 is given in GenBank Acc. No. NP 036370, disclosed as being composed of 747 amino acid residues, is shown in Table 2 using the conventional one-letter amino acid code (International Union Of Biochemistry And Molecular Biology, Recommendations on Biochemical & Organic Nomenclature, Symbols & Terminology etc., Part 1, Section A: Amino-Acid Nomenclature, Section 3AA-1. Names Of Common Alpha-Amino Acids, http://www.chem.qmul.ac.uk/iubmb/ and J. Biol. Chem., 1985, 260, 14-42).

TABLE 2
Amino Acid Sequence of Human SIRT1.

| | | | | | | | |
|---|---|---|---|---|---|---|---|
| 1 | madeaalalq | pggspsaaga | dreaasspag | eplrkrprrd | gpglerspge | pggaaperev | (SEQ ID NO:1) |
| 61 | paaargcpga | aaaalwreae | aeaaaaggeq | eaqataaage | gdngpglqgp | sreppladnl | |
| 121 | ydeddddege | eeeeaaaaai | gyrdnllfgd | eiitngfhsc | esdeedrash | asssdwtprp | |
| 181 | rigpytfvqq | hlmigtdprt | ilkdllpeti | pppelddmtl | wqivinilse | ppkrkkrkdi | |
| 241 | ntiedavkll | qeckkiivlt | gagvsvscgi | pdfrsrdgiy | arlavdfpdl | pdpqamfdie | |
| 301 | yfrkdprpff | kfakeiypgq | fqpslchkfi | alsdkegkll | rnytqnidtl | eqvagiqrii | |
| 361 | qchgsfatas | clickykvdc | eavrgdifnq | vvprcprcpa | deplaimkpe | ivffgenlpe | |
| 421 | qfhramkydk | devdllivig | sslkvrpval | ipssiphevp | qilinreplp | hlhfdvellg | |
| 481 | dcdviinelc | hrlggeyakl | ccnpvklsei | tekpprtqke | laylselppt | plhvsedsss | |
| 541 | pertsppdss | vivtlldqaa | ksnddldvse | skgcmeekpq | evqtsrnves | iaeqmenpdl | |
| 601 | knvgsstgek | nertsvagtv | rkcwpnrvak | eqisrrldgn | qylflppnry | ifhgaevysd | |
| 661 | seddvlssss | cgsnsdsgtc | qspsleepme | deseieefyn | gledepdvpe | raggagfgtd | |
| 721 | gddqeainea | isvkqevtdm | nypsnks | | | | |

In general, an “SIRT1 polypeptide” employed in the methods and compositions of the present invention, includes wild type human SIRT1 such as represented in Table 2, as well as wild type vertebrate orthologs thereof, and domains, motifs and fragments thereof. In addition, an “SIRT1 polypeptide” additionally includes recombinant mutant polypeptides, domains, motifs and fragments in which at least one amino acid residue has been changed to a different amino acid residue; or one or more residues may be deleted; or one or more residues may be inserted between neighboring residues in an original sequence. A mutant or variant SIRT1 polypeptide may have from 1 amino acid residue up to 1% of the residues changed, or up to 2%, or up to 5%, or up to 8%, or up to 10%, or up to 15%, or up to 20%, or somewhat higher percent, of the residues changed from a wild type or reference sequence. The recombinant mutant or variant polypeptides, domains, motifs and fragments of SIRT1 are used in the present methods and compositions as long as they demonstrably exhibit at least one biological activity or function of wild type SIRT1. Possession of a biological activity or function may be determined by a worker of skill in the fields related to the present invention, including, by way of nonlimiting example, molecular biology, cell biology, pathology, clinical medicine and the like. Such workers of skill in the fields of the invention may assay recombinant mutant SIRT1 polypeptides, domains, motifs and fragments at least by methods described in the Examples of the present invention.

It will be recognized in the art that an amino acid sequence of an SIRT1 polypeptide can be varied without significant effect on the structure or function of the protein. If such differences in sequence are contemplated, it should be remembered that there will be certain areas on the protein that are important for its activity. In general, it is possible to replace residues that form the tertiary structure, provided that residues providing a similar function are used. In other instances, the type of residue may be completely unimportant if the alteration occurs at a non-important region of the protein.

Thus, the invention further includes variations of an SIRT1 polypeptide that show substantial SIRT1 polypeptide activity or which include regions of SIRT1 protein such as the protein portions discussed below. Such mutants include deletions, insertions, inversions, repeats, and structurally or functionally conservative substitutions (for example, substituting one hydrophilic residue for another, or a hydrophobic residue for another). Such amino acid substitutions will generally have little effect on activity.

Examples of conservative substitutions are the replacements, one for another, among the aliphatic amino acids Ala, Val, Leu, Ile and Met; interchange of the hydroxyl residues Ser and Thr; exchange of the acidic residues Asp and Glu; substitution between the amide residues Asn and Gln; exchange of the basic residues His, Lys and Arg; and replacements among the aromatic residues Phe, Tyr and Trp. Additionally variant forms of an SIRT1 polypeptide may be one in which the polypeptide is fused with another compound, such as a compound to increase the half-life of the polypeptide (for example, polyethylene glycol), or one in which additional amino acids are fused to the polypeptide, such as an IgG Fc fusion region peptide or leader or secretory sequence or a sequence which is employed for purification of the polypeptide. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of an SIRT1 protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol. 2: 331-340 (1967); Robbins et al., Diabetes 36: 838-845 (1987); Cleland et al., Crit. Rev. Therapeutic Drug Carrier Systems 10: 307-377 (1993)).

As indicated, changes are preferably of an inconsequential nature, such as introduction of conservative amino acid substitutions that do not significantly affect the folding or activity of the SIRT1 protein. Table 3 provides nonlimiting examples of conservative substitutions contemplated herein. In Table 3 a given amino acid residue, since it may have more than chemical or physical attribute, may appear in one, or in more than one, class.

TABLE 3
Examples of Structural or Functional Conservative
Amino Acid Substitutions.

| | |
|---|---|
| Aromatic | Phenylalanine Tryptophan Tyrosine Histidine |
| Hydrophobic | Leucine Isoleucine Valine Alanine Methionine |
| | Phenylalanine |
| Polar | Glutamine Asparagine Serine Threonine Cysteine Tyrosine |
| | Tryptophan Histidine |
| Basic | Arginine Lysine Histidine |
| Acidic | Aspartic Acid Glutamic Acid |
| Amphipathic | Alanine Serine Threonine Glycine Proline |

Amino acid residues in an SIRT1 protein of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity. Sites that are critical for ligand-receptor binding can also be determined by structural analysis such as crystallization, nuclear magnetic resonance or photoaffinity labeling (Smith et al, J. Mol. Biol. 224: 899-904 (1992) and de Vos et al. Science 255: 306-312 (1992)).

The polypeptides of the present invention include a full length polypeptide including the leader; and a mature polypeptide. As used herein, a “mature” form of a polypeptide or protein may be a final translation product of the corresponding nucleotide sequence within the vertebrate cell, and is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Important embodiments of a variant SIRT1 or a fragment of any SIRT1 polypeptide possess at least one biological activity, such as an enzymatic activity, or a biological function, such as an effect on a cell, or an effect on a signaling pathway, or an effect on the level of expression in a cell of a non-SIRT1 polypeptide. Other important embodiments of a fragment of any SIRT1 polypeptide serve as haptens or immunogens in stimulating production of an anti-SIRT1 antibody (see below).

Determining Similarity Between Two Or More Sequences

To determine the percent similarity of two amino acid sequences or of two nucleic acid sequences, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (i.e., as used herein amino acid or nucleic acid “identity” is equivalent to amino acid or nucleic acid “homology”).

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T or U, C, G, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

“Identity,” as known in the art, is a relationship between two or more polypeptide sequences or two or more polynucleotide sequences, as determined by, comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk. A. M., ed., Oxford University Press, New York, 1988; Biocomputing: Informatics and Genome Projects, Smith, D. W., ed., Academic Press, New York, 1993; Computer Analysis of Sequence Data, Part I. Griffin, A. M., and Griffin, H. G., eds. Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press. New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devercux, J., et al., Nucleic Acids Research 12(1): 387 (1984)), BLASTP, BLASTN, and FASTA (Atschul, S. F. et al., J. Mol. Biol. 215: 403410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following: Algorithm: Needleman and Wunsch, J. Mol. Biol. 48: 443-453 (1970).

Comparison matrix: BLOSSUM62 from Hentikoff and Hentikoff, Proc. Natl. Acad. Sci. USA. 89: 10915-10919 (1992).

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm: Needleman and Wunsch. J. Mol. Biol. 48: 443453 (1970).

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided below.

Polynucleotide embodiments further include an isolated polynucleotide that includes a polynucleotide sequence having at least a 50, 60, 70, 80, 85, 90, 95, 97 or 100% identity to a reference nucleotide sequence such as the wild type sequence of Table 4, wherein said polynucleotide sequence may be identical to the reference sequence, or may include up to a certain integer number of nucleotide alterations as compared to the reference sequence, wherein said alterations are selected from the group consisting of at least one nucleotide deletion, substitution including transition and transversion, or insertion, and wherein said alterations may occur at the 5′ or 3′ terminal positions of the reference nucleotide sequence or anywhere between those terminal positions interspersed either individually among the nucleotides in the reference sequence or in one or more contiguous groups within the reference sequence, and wherein said number of nucleotide alterations is determined by multiplying the total number of nucleotides in the reference polynucleotide sequence by the integer defining the percent identity divided by 100 and then subtracting that product from said total number of nucleotides in the reference polynucleotide sequence, or:
nₙ≦xₙ−(xₙ*y)
wherein nₙ is the number of nucleotide alterations, xₙ is the total number of nucleotides in the reference polynucleotide sequence, y is 0.50 for 50%, 0.60 for 60%, 0.70 for 70%, 0.80 for 80%, 0.85 for 85% 0.90 for 90%, 0.95 for 95% 0.97 for 97% or 1.00 for 100%, and * is the symbol for the multiplication operator, and wherein any non-integer product of xₙ and y is rounded down to the nearest integer prior to subtracting it from xₙ. Alterations of a polynucleotide sequence encoding the polypeptide of wild type SIRT2 of Table 2 may create nonsense, missense or frameshift mutations in this coding sequence and thereby alter the polypeptide encoded by the polynucleotide following such alterations.

Additionally the BLAST alignment tool is useful for detecting similarities and percent identity between two sequences. BLAST is available on the World Wide Web at the National Center for Biotechnology Information site. References describing BLAST analysis include Madden, T. L., Tatusov, R. L. & Zhang, J. (1996) “Applications of network BLAST server” Meth. Enzymol. 266: 131-141; Altschul, S. F., Madden, T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25: 3389-3402; and Zhang, J. & Madden, T. L. (1997) “PowerBLAST: A new network BLAST application for interactive or automated sequence analysis and annotation.” Genome Res. 7: 649-656.

The polypeptides of the present invention are preferably provided in an isolated form. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell. For example, a recombinantly produced version of an SIRT1 polypeptide can be substantially purified by the one-step method described in Smith and Johnson, Gene 67: 31-40 (1988). Isolated SIRT1 polypeptides may be used as immunogens to stimulate the production of anti-SIRT1 antibodies.

Nucleic Acids

As used herein, the term “SIRT1 polynucleotide” or “SIRT1 nucleic acid”, or related terms and phrases, relates to any polynucleotide that encodes any SIRT1 polypeptide as described herein. In general, any nucleotide sequence that encodes an SIRT1 polypeptide described above is encompassed within the present invention. In some embodiments, a nucleic acid encoding a polypeptide having the amino acid sequence of a human SIRT1 shown in Table 2 includes a coding sequence of the mRNA nucleic acid sequence disclosed in GenBank Acc. No. NM_{—}012238, shown in Table 4, or a fragment thereof. In Table 4, the coding sequence extends from position 54 to position 2297.

TABLE 4

| | | | | | | | |
|---|---|---|---|---|---|---|---|
| 1 | gtcgagcggg | agcagaggag | gcgagggagg | agggccagag | aggcagttgg | aagatggcgg | (SEQ ID NO:2) |
| 61 | acgaggcggc | cctcgccctt | cagcccggcg | gctccccctc | ggcggcgggg | gccgacaggg | |
| 121 | aggccgcgtc | gtcccccgcc | ggggagccgc | tccgcaagag | gccgcggaga | gatggtcccg | |
| 181 | gcctcgagcg | gagcccgggc | gagcccggtg | gggcggcccc | agagcgtgag | gtgccggcgg | |
| 241 | cggccagggg | ctgcccgggt | gcggcggcgg | cggcgctgtg | gcgggaggcg | gaggcagagg | |
| 301 | cggcggcggc | aggcggggag | caagaggccc | aggcgactgc | ggcggctggg | gaaggagaca | |
| 361 | atgggccggg | cctgcagggc | ccatctcggg | agccaccgct | ggccgacaac | ttgtacgacg | |
| 421 | aagacgacga | cgacgagggc | gaggaggagg | aagaggcggc | ggcggcggcg | attgggtacc | |
| 481 | gagataacct | tctgttcggt | gatgaaatta | tcactaatgg | ttttcattcc | tgtgaaagtg | |
| 541 | atgaggagga | tagagcctca | catgcaagct | ctagtgactg | gactccaagg | ccacggatag | |
| 601 | gtccatatac | ttttgttcag | caacatctta | tgattggcac | agatcctcga | acaattctta | |
| 661 | aagatttatt | gccggaaaca | atacctccac | ctgagttgga | tgatatgaca | ctgtggcaga | |
| 721 | ttgttattaa | tatcctttca | gaaccaccaa | aaaggaaaaa | aagaaaagat | attaatacaa | |
| 781 | ttgaagatgc | tgtgaaatta | ctgcaagagt | gcaaaaaaat | tatagttcta | actggagctg | |
| 841 | gggtgtctgt | ttcatgtgga | atacctgact | tcaggtcaag | ggatggtatt | tatgctcgcc | |
| 901 | ttgctgtaga | cttcccagat | cttccagatc | ctcaagcgat | gtttgatatt | gaatatttca | |
| 961 | gaaaagatcc | aagaccattc | ttcaagtttg | caaaggaaat | atatcctgga | caattccagc | |
| 1021 | catctctctg | tcacaaattc | atagccttgt | cagataagga | aggaaaacta | cttcgcaact | |
| 1081 | atacccagaa | catagacacg | ctggaacagg | ttgcgggaat | ccaaaggata | attcagtgtc | |
| 1141 | atggttcctt | tgcaacagca | tcttgcctga | tttgtaaata | caaagttgac | tgtgaagctg | |
| 1201 | tacgaggaga | tatttttaat | caggtagttc | ctcgatgtcc | taggtgccca | gctgatgaac | |
| 1261 | cgcttgctat | catgaaacca | gagattgtgt | tttttggtga | aaatttacca | gaacagtttc | |
| 1321 | atagagccat | gaagtatgac | aaagatgaag | ttgacctcct | cattgttatt | gggtcttccc | |
| 1381 | tcaaagtaag | accagtagca | ctaattccaa | gttccatacc | ccatgaagtg | cctcagatat | |
| 1441 | taattaatag | agaacctttg | cctcatctgc | attttgatgt | agagcttctt | ggagactgtg | |
| 1501 | atgtcataat | taatgaattg | tgtcataggt | taggtggtga | atatgccaaa | ctttgctgta | |
| 1561 | accctgtaaa | gctttcagaa | attactgaaa | aacctccacg | aacacaaaaa | gaattggctt | |
| 1621 | atttgtcaga | gttgccaccc | acacctcttc | atgtttcaga | agactcaagt | tcaccagaaa | |
| 1681 | gaacttcacc | accagattct | tcagtgattg | tcacactttt | agaccaagca | gctaagagta | |
| 1741 | atgatgattt | agatgtgtct | gaatcaaaag | gttgtatgga | agaaaaacca | caggaagtac | |
| 1801 | aaacttctag | gaatgttgaa | agtattgctg | aacagatgga | aaatccggat | ttgaagaatg | |
| 1861 | ttggttctag | tactggggag | aaaaatgaaa | gaacttcagt | ggctggaaca | gtgagaaaat | |
| 1921 | gctggcctaa | tagagtggca | aaggagcaga | ttagtaggcg | gcttgatggt | aatcagtatc | |
| 1981 | tgtttttgcc | accaaatcgt | tacattttcc | atggcgctga | ggtatattca | gactctgaag | |
| 2041 | atgacgtctt | atcctctagt | tcttgtggca | gtaacagtga | tagtgggaca | tgccagagtc | |
| 2101 | caagtttaga | agaacccatg | gaggatgaaa | gtgaaattga | agaattctac | aatggcttag | |
| 2161 | aagatgagcc | tgatgttcca | gagagagctg | gaggagctgg | atttgggact | gatggagatg | |
| 2221 | atcaagaggc | aattaatgaa | gctatatctg | tgaaacagga | agtaacagac | atgaactatc | |
| 2281 | catcaaacaa | atcatagtgt | aataattgtg | caggtacagg | aattgttcca | ccagcattag | |
| 2341 | gaactttagc | atgtcaaaat | gaatgtttac | ttgtgaactc | gatagagcaa | ggaaaccaga | |
| 2401 | aaggtgtaat | atttataggt | tggtaaaata | gattgttttt | catggataat | ttttaacttc | |
| 2461 | attatttctg | tacttgtaca | aactcaacac | taactttttt | ttttttaaaa | aaaaaaaggt | |
| 2521 | actaagtatc | ttcaatcagc | tgttggtcaa | gactaacttt | cttttaaagg | ttcatttgta | |
| 2581 | tgataaattc | atatgtgtat | atataatttt | ttttgttttg | tctagtgagt | ttcaacattt | |
| 2641 | ttaaagtttt | caaaaagcca | tcggaatgtt | aaattaatgt | aaagggacag | ctaatctaga | |
| 2701 | ccaaagaatg | gtattttcac | ttttctttgt | aacattgaat | ggtttgaagt | actcaaaatc | |
| 2761 | tgttacgcta | aacttttgat | tctttaacac | aattattttt | aaacactggc | attttccaaa | |
| 2821 | actgtggcag | ctaacttttt | aaaatctcaa | atgacatgca | gtgtgagtag | aaggaagtca | |
| 2881 | acaatatgtg | gggagagcac | tcggttgtct | ttacttttaa | aagtaatact | tggtgctaag | |
| 2941 | aatttcagga | ttattgtatt | tacgttcaaa | tgaagatggc | ttttgtactt | cctgtggaca | |
| 3001 | tgtagtaatg | tctatattgg | ctcataaaac | taacctgaaa | aacaaataaa | tgctttggaa | |
| 3061 | atgtttcagt | tgctttagaa | acattagtgc | ctgcctggat | ccccttagtt | ttgaaatatt | |
| 3121 | tgccattgtt | gtttaaatac | ctatcactgt | ggtagagctt | gcattgatct | tttccacaag | |
| 3181 | tattaaactg | ccaaaatgtg | aatatgcaaa | gcctttctga | atctataata | atggtacttc | |
| 3241 | tactggggag | agtgtaatat | tttggactgc | tgttttccat | taatgaggag | agcaacaggc | |
| 3301 | ccctgattat | acagttccaa | agtaataaga | tgttaattgt | aattcagcca | gaaagtacat | |
| 3361 | gtctcccatt | gggaggattt | ggtgttaaat | accaaactgc | tagccctagt | attatggaga | |
| 3421 | tgaacatgat | gatgtaactt | gtaatagcag | aatagttaat | gaatgaaact | agttcttata | |
| 3481 | atttatcttt | atttaaaagc | ttagcctgcc | ttaaaactag | agatcaactt | tctcagctgc | |
| 3541 | aaaagcttct | agtctttcaa | gaagttcata | ctttatgaaa | ttgcacagta | agcatttatt | |
| 3601 | tttcagacca | tttttgaaca | tcactcctaa | attaataaag | tattcctctg | ttgctttagt | |
| 3661 | atttattaca | ataaaaaggg | tttgaaatat | agctgttctt | tatgcataaa | acacccagct | |
| 3721 | aggaccatta | ctgccagaga | aaaaaatcgt | attgaatggc | catttcccta | cttataagat | |
| 3781 | gtctcaatct | gaatttattt | ggctacacta | aagaatgcag | tatatttagt | tttccatttg | |
| 3841 | catgatgttt | gtgtgctata | gatgatattt | taaattgaaa | agtttgtttt | aaattatttt | |
| 3901 | tacagtgaag | actgttttca | gctcttttta | tattgtacat | agtcttttat | gtaatttact | |
| 3961 | ggcatatgtt | ttgtagactg | tttaatgact | ggatatcttc | cttcaacttt | tgaaatacaa | |
| 4021 | aaccagtgtt | ttttacttgt | acactgtttt | aaagtctatt | aaaattgtca | tttgactttt | |
| 4081 | ttctgttaaa | aaaaaaaaaa | aaaaaaa | | | | |

Additionally, the invention includes SIRT1 polynucleotides that are mutant or variant nucleic acids of the sequence shown in Table 4, or a fragment thereof, any of whose bases may be changed from the disclosed sequence while still encoding a polypeptide that maintains its SIRT1 protein-like activities and physiological functions. An SIRT1 mutant or variant polynucleotide encodes a mutant or variant SIRT1 polypeptide that may have from 1 amino acid residue up to 1% of the residues changed, or up to 2%, or up to 5%, or up to 8%, or up to 10%, or up to 15%, or up to 20%, or somewhat higher percent, of the residues changed from a wild type or reference sequence. By “nucleic acid” or “polynucleotide” is meant a DNA, an RNA, a DNA or RNA including one or more modified nucleotides or modified pentose phosphate backbone structures, a polypeptide-nucleic acid, and similar constructs that preserve the coding properties of the sequence of bases included in the construct. The invention further includes the complement of the nucleic acid sequence of any SIRT1 encoding sequence, including fragments, derivatives, analogs and homolog thereof. The invention additionally includes nucleic acids or nucleic acid fragments, or complements thereto, whose structures include chemical modifications.

Also included are SIRT1 nucleic acid fragments. A nucleic acid fragment may encode a fragment of an SIRT1 polypeptide. In addition SIRT1 nucleic fragments may be used as hybridization probes to identify SIRT1 protein-encoding nucleic acids (e.g., SIRT1 mRNA) and fragments for use as polymerase chain reaction (PCR) primers for the amplification or mutation of SIRT1 nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source (although they may be prepared by chemical synthesis as well), are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated SIRT1 nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of Table 4, or a complement of any of this nucleotide sequence, can be isolated using standard molecular biology techniques and the sequence information provided herein. Using all or a portion of the nucleic acid sequence of Table 4 as a hybridization probe, SIRT1 nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.).

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SIRT1 nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide that includes a nucleic acid molecule less than 100 nt in length would further comprise at lease 6 contiguous nucleotides of Table 4, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in Table 4. In another embodiment, an isolated nucleic acid molecule of the invention comprises a nucleic acid molecule that is a complement of the nucleotide sequence shown in Table 4, or a portion of this nucleotide sequence. A nucleic acid molecule that is complementary to the nucleotide sequence shown in is one that is sufficiently complementary to the nucleotide sequence shown in Table 4 that it can hydrogen bond with little or no mismatches to the nucleotide sequence shown in of Table 4, thereby forming a stable duplex.

Moreover, the nucleic acid molecule of the invention can contain only a portion of the nucleic acid sequence of Table 4, e.g., a fragment that can be used as a probe or primer, or a fragment encoding a biologically active portion of an SIRT1 protein. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type.

Derivatives and analogs of polynucleotides and polypeptides may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99/o) over a nucleic acid or amino acid sequence of identical size or when compared to an aligned sequence using methods described in detail below.

“Percent identity”, or “percent similarity”, or “homology”, or variations thereof, when used to characterize a nucleic acid sequence or an amino acid sequence, refer to sequences characterized by a similarity at the nucleotide level or amino acid level as discussed above. Similar nucleotide sequences encode those sequences coding for isoforms of an SIRT1 polypeptide. Isoforms can be expressed in different tissues of the same organism as a result of, for example, alternative splicing of RNA. Alternatively, isoforms can be encoded by different genes. In the present invention, similar nucleotide sequences include nucleotide sequences encoding for an SIRT1 polypeptide of species other than humans, including, but not limited to, mammals, and thus can include, e.g., mouse, rat, rabbit, dog, cat cow, horse, and other organisms. Similar nucleotide sequences also include, but are not limited to, naturally occurring allelic variations and mutations of the nucleotide sequences set forth herein. A similar nucleotide sequence does not, however, include the nucleotide sequence encoding a human SIRT1 protein. Similar nucleic acid sequences include those nucleic acid sequences that encode conservative amino acid substitutions (see below) in any SIRT1 polypeptide as well as a polypeptide having SIRT1 protein activity. Biological activities of the SIRT1 proteins are described herein.

The nucleotide sequence determined from the cloning of the human SIRT1 gene allows for the generation of probes and primers designed for use in identifying the cell types disclosed and/or cloning SIRT1 homologues in other cell types, e.g., from other tissues, as well as SIRT1 homologues from other mammals. The probe/primer typically comprises a substantially purified oligonucleotide. The oligonucleotide typically comprises a region of nucleotide sequence that hybridizes with high specificity under suitable conditions to at least about 12, 25, 50, 100, 150, 200, 250, 300, 350 or 400 or more consecutive sense strand nucleotide sequence of the nucleotide sequence of Table 4; or an anti-sense strand nucleotide sequence of Table 4; or of a naturally occurring mutant of Table 4.

Anti-SIRT1 Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an Fab expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species. Any antibody disclosed herein binds “immunospecifically” to its cognate antigen. By immunospecific binding is meant that an antibody raised by challenging a host with a particular immunogen binds to a molecule such as an antigen that includes the immunogenic moiety with a high affinity, and binds with only a weak affinity or not at all to non-immunogen-containing molecules. As used in this definition, high affinity means having a dissociation constant less than about 1×10⁻⁶ M, and weak affinity means having a dissociation constant higher than about 1×10⁻⁶ M.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in Table 2, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of the SIRT1 protein that is located on the surface of the protein, e.g., a hydrophilic region. A hydrophobicity analysis of the human SIRT1 protein sequence will indicate which regions of a growth promoting polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256: 495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor: J. Immunol., 133: 3001 (1984); Brodeur et al.: Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem. 107: 220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

SIRT1 Recombinant Vectors and Host Cells

Another aspect of the invention pertains to vectors, preferably expression vectors, containing a nucleic acid encoding SIRT1 protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SIRT1 proteins, mutant forms of the SIRT1 protein, fusion proteins, etc.).

The recombinant expression vectors of the invention can be designed for expression of the SIRT1 protein in prokaryotic or eukaryotic cells. For example, the SIRT1 protein can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20: 2111-2118). Such alteration of nucleic acid sequences of the invention can be carried out by standard DNA synthesis techniques.

In another embodiment, the SIRT1 expression vector is a yeast expression vector. Examples of vectors for expression in yeast S. cerivisae include pYepSec1 (Baldari, et al., (1987) EMBO J 6: 229-234), pMFa (Kurjan and Herskowitz, (1982) Cell 30: 933-943), pJRY88 (Schultz et al., (1987) Gene 54: 113-123), pYES2 (Invitrogen Corporation, San Diego, Calif.), and picZ (InVitrogen Corp, San Diego, Calif.).

Alternatively, the SIRT1 protein can be expressed in insect cells using baculovirus expression vectors. Baculovirus vectors available for expression of proteins in cultured insect cells (e.g., SF9 cells) include the pAc series (Smith et al. (1983) Mol Cell Biol 3: 2156-2165) and the pVL series (Lucklow and Summers (1989) Virology 170: 31-39).

In yet another embodiment, a nucleic acid of the invention is expressed in mammalian cells using a mammalian expression vector. Examples of mammalian expression vectors include pCDM8 (Seed (1987) Nature 329: 840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1: 268-277), lymphoid-specific promoters (Calame and Eaton (1988) Adv Immunol 43: 235-275), in particular promoters of T cell receptors (Winoto and Baltimore (1989) EMBO J 8: 729-733) and immunoglobulins (Banerji et al. (1983) Cell 33: 729-740; Queen and Baltimore (1983) Cell 33: 741-748), neuron-specific promoters (e.g., the neurofilament promoter; Byrne and Ruddle (1989) PNAS 86: 5473-5477), pancreas-specific promoters (Edlund et al. (1985) Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249: 374-379) and the α-fetoprotein promoter (Campes and Tilghman (1989) Genes Dev 3: 537-546).

The invention further provides a recombinant expression vector that includes a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to a SIRT1 mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the SIRT1 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (2001), Ausubel et al. (2002), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the growth promoter or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the SIRT1 protein. Accordingly, the invention further provides methods for producing the SIRT1 protein using the host cells of the invention. In one embodiment, the method comprises culturing the host cell of invention (into which a recombinant expression vector encoding the SIRT1 protein has been introduced) in a suitable medium such that the SIRT1 protein is produced. In another embodiment, the method further comprises isolating the SIRT1 protein from the medium or the host cell.

Transfection of a vertebrate cell can further be accomplished using recombinant vectors which include, but are not limited, to adenovirus, adeno-associated virus, and retrovirus vectors, in addition to other particles that introduce DNA into cells, such as liposomes. Techniques such as those described above can be utilized for the introduction of any SIRT1 polypeptide encoding nucleotide sequences into vertebrate cells. For example, for transfection of mammalian cells, a number of viral-based expression systems may be utilized. In cases where an adenovirus is used as an expression vector, the SIRT1 nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing an SIRT1 product in infected hosts (e.g., See Logan & Shenk, 1984, Proc. Natl. Acad. Sci. USA 81: 3655-3659). In cases where only a portion of an SIRT1 coding sequence is inserted, exogenous translational control signals, including, perhaps, the ATG initiation codon, must be provided. These exogenous translational control signals and initiation codons can be of a variety of origins, both natural and synthetic. The efficiency of expression can be enhanced by the inclusion of appropriate transcription enhancer elements, transcription terminators, etc. (See Bitter et al., 1987, Methods in Enzymol. 153: 516-544).

Therapeutic Treatment

Certain pathologies and medical conditions are believed to respond favorably to the expression of heterologous SIRT1 in the cells of a subject. Accordingly, the present invention discloses a method of treating a pathology, a disease or a medical condition in a subject, wherein the pathology responds to an SIRT1 polypeptide. The method includes administering a nucleic acid encoding an SIRT1 polypeptide to the subject in an amount effective to attenuate or ameliorate the pathology. Attenuating a pathology signifies that a trend of worsening symptomology is abated to a slower or more gentle trend of worsening. Ameliorating a pathology signifies an actual improvement in the patient, such that the signs and indications of the pathology diminish, and the patient improves toward better health. In important implementations of this method the pathology is chosen from among myocardial infarction, cerebrovascular stroke, a kidney disease, a neurological disease, wound healing, healing from surgical incisions, bone healing, preservation of dermal, epidermal, mucosal epithelial surfaces, and the like. In advantageous embodiments of the method of treating a pathology the subject is a human.

In various embodiments of the methods of treatment described herein, a nucleic acid encoding an SIRT1 polypeptide, a variant thereof, or a fragment thereof, may be administered to a subject in any of a variety of compositions that ensure efficient delivery of the nucleic acid sequence into cells, including delivery into the cells of a subject.

Treatment of a subject with an SIRT1 nucleic acid sequence can be accomplished by administering a suitable nucleic acid, plasmid, vector, viral vector, liposomal or similar composition that is effective to introduce the SIRT1 nucleic acid sequence into a vertebrate cell. Transfection of nucleic acids may be assisted with the use of cationic amphiphiles (U.S. Pat. No. 6,503,945 and references disclosed therein). Ex vivo retroviral gene therapy is described, for example, in Hacein-Bey-Abina et al. (2003, Science 302: 415-419). Methods for therapeutic introduction of a transgene into a subject are discussed in “Gene Transfer Methods: Introducing DNA Into Living Cells and Organisms” P. A. Norton and L. F. Steel, Eaton Publishing, 2000. Approaches to the therapeutic introduction of transgenes into cells and organisms are provided in “Gene Therapy Protocols” Paul D. Robbins (Ed.), Humana Press (1997).

Transgenic Animals

The SIRT1-transfected cells of the invention can also be used to produce nonhuman transgenic animals. For example, in one embodiment, an SIRT1-transfected cell of the invention is a fertilized oocyte or an embryonic stem cell into which SIRT1 protein-coding sequences have been introduced. Such cells can then be used to create non-human transgenic animals in which exogenous SIRT1 protein sequences have been introduced into the animal's genome or homologous recombinant animals in which endogenous SIRT1 protein sequences have been altered. Such animals are useful for studying the function and/or activity of the SIRT1 proteins and for identifying and/or evaluating modulators of SIRT1 protein activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, etc. A transgene is exogenous DNA that is stably integrated into the genome of a cell from which a transgenic animal develops, thereby directing the expression of an encoded gene product in one or more cell types or tissues of the transgenic animal. As used herein, a “homologous recombinant animal” is a non-human animal, preferably a mammal, more preferably a mouse, in which an endogenous SIRT1 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing SIRT1 protein-encoding nucleic acid into the male pronuclei of a fertilized oocyte, e.g., by microinjection, retroviral infection, and allowing the oocyte to develop in a pseudopregnant female foster animal. The human SIRT1 DNA sequence of Table 4, or any SIRT1 polynucleotide of the invention can be introduced as a transgene into the genome of a non-human animal. Alternatively, a nonhuman homologue of the human SIRT1 gene, such as a mouse SIRT1 gene, can be isolated based on hybridization to the human SIRT1 cDNA (described further above) and used as a transgene. Intronic sequences and polyadenylation signals can also be included in the transgene to increase the efficiency of expression of the transgene. A tissue-specific regulatory sequence(s) can be operably linked to the SIRT1 transgene to direct expression of SIRT1 protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the SIRT1 transgene in its genome and/or expression of SIRT1 mRNA in tissues or cells of the animals. A transgenic founder animal can then be used to breed additional animals carrying the transgene. Moreover, transgenic animals carrying a transgene encoding an SIRT1 protein can further be bred to other transgenic animals carrying other transgenes.

To create a homologous recombinant animal, a vector is prepared which contains at least a portion of an SIRT1 gene into which a deletion, addition or substitution has been introduced to thereby alter, e.g., functionally disrupt, the SIRT1 gene. The SIRT1 gene can be a human gene (e.g., Table 4), but more preferably, is a non-human homologue of a human SIRT1 gene. For example, a mouse homologue of human SIRT1 gene of Table 4 can be used to construct a homologous recombination vector suitable for altering an endogenous SIRT1 gene in the mouse genome. In one embodiment, the vector is designed such that, upon homologous recombination, the endogenous SIRT1 gene is functionally disrupted (i.e., no longer encodes a functional protein; also referred to as a “knock out” vector).

Alternatively, the vector can be designed such that, upon homologous recombination, the endogenous SIRT1 gene is mutated or otherwise altered but still encodes functional protein (e.g., the upstream regulatory region can be altered to thereby alter the expression of the endogenous SIRT1 protein). In the homologous recombination vector, the altered portion of the SIRT1 gene is flanked at its 5′ and 3′ ends by additional nucleic acid of the SIRT1 gene to allow for homologous recombination to occur between the exogenous SIRT1 protein gene carried by the vector and an endogenous SIRT1 protein gene in an embryonic stem cell. The additional flanking SIRT1 protein nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51: 503 for a description of homologous recombination vectors. The vector is introduced into an embryonic stem cell line (e.g., by electroporation) and cells in which the introduced SIRT1 protein gene has homologously recombined with the endogenous SIRT1 protein gene are selected (see e.g., Li et al. (1992) Cell 69: 915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2: 823-829; PCT International Publication Nos.: WO 90/1184; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89: 6232-6236. Another example of a recombinase system is the FLP recombinase system of Saccharomyces cerevisiae (O'Gorman et al. (1991) Science 251: 181-185. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385: 810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Methods for generating transgenic animals are additionally discussed in “Gene Transfer Methods: Introducing DNA Into Living Cells and Organisms” P. A. Norton and L. F. Steel, Eaton Publishing, 2000; and in “Transgenesis Techniques: Principles and Protocols”, 2nd ed., A. R. Clarke, Humana Press, 2002.

### EXAMPLES

### Reference Example 1

### Cloning of SIRT1

The SIRT1 gene was obtained by PCR amplification using the following primers:

| | |
|---|---|
| 5′ primer-1: | |
| GGATCCACCATGGCGGACGAGGCGGCCCTCGCC | (SEQ ID NO:3) |
| and | |
| 3′ primer-2: | |
| GTCTAGAGTGGAACAATTCCTGTACCTGCAC | (SEQ ID NO:4) |
| (see Vaziri et al., 2001). | |

PCR was carried out using a human spleen Marathon-ready cDNA library (Cat. No. 639312 Clontech; BD Biosciences Clontech, Palo Alto, Calif.). This provided a majority of the SIRT1 cDNA including the C terminus. In order to obtain the 5′ end of SIRT1, which is GC-rich in nature, a human genomic clone (Accession number: AL133551, clone RP11-57G10) was used as a template to obtain the 5′ end. 10 PCR cycles were carried out using PfuTurbo® DNA Polymerase (Stratagene, La Jolla, Calif.) under the following conditions: denaturation at 98° C., and addition of IM betaine and 10% DMSO to the Stratagene pfu buffer. The primers were Primer-1 (above) and

| | |
|---|---|
| Primer-3: | |
| GAGGAGGAGATGCGCAGTTCCGGCCGCCC. | (SEQ ID NO:5) |

The PCR product was cloned into pcr4blunt-TOPO (Invitrogen, Carlsbad, Calif.) and sequenced. The resulting exon, exon-1 on SIRT1, was used to complete the sequence of the SIRT1 amplicon obtained using 5′ Primer-1 and 3′ Primer-2.

### Reference Example 2

### Preparation of the Mutant SIRT1-HY

A site-specific mutant intended to eliminate the deacetylase activity of SIRT1 was designed (see Vaziri et al., 2001). To prepare the mutant, the PCR overlap primer method was used to create a point mutation (CAT to TAT) at codon 363, converting residue 363 from histidine (H) to tyrosine (Y).

### Reference Example 3

### Construction of Expression Plasmids

A BamHI/SnaBI fragment of SIRT1 cDNA isolated from a cDNA library (Clontech) as in Reference Example 1 was inserted into pBabe-Y-Puro (see Vaziri et al., 2001), the resulting plasmid was called pYESir2-puro. Similarly a BamHI/SnaBI fragment of SIRT1 that was mutated at residue 363 from histidine (H) to tyrosine (Y) by site-directed mutagenesis (Stratagene) (Reference Example 2) was used to create the retroviral vector pYESir2HY.

### Example 1

### Assessment of SIRT1 in Human Cells at Different PDL Values

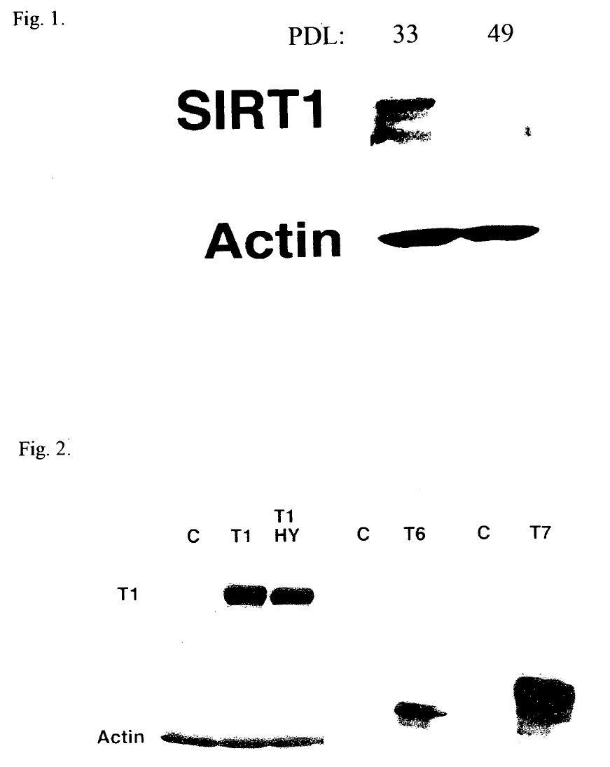

WI-38 cells (The Coriell Institute for Medical Research, Camden, N.J.) are a human diploid cell line derived from normal embryonic lung tissue. WI-38 cells have a lifetime of 50±10 population doublings. WI-38 cells were cultured in minimum essential medium (MEM) for an extended time, during which the PDL was tracked. At PDL values of 33 and 49 the amount of SIRT1 protein in the cells was assessed by Western blot of an SDS-PAGE electrophoretogram The antibody probe was a rabbit polyclonal anti-SIRT1 antibody prepared using the peptide DEEDRASHASS (SIRT1 residues 164-173, i.e., residues 164-173 of SEQ ID NO:1), and was kindly provided by Dr. Namjin Chung, Dept. of Biology, Massachusetts Institute of Technology, Cambridge, Mass. The results are shown in FIG. 1, together with blot detection of actin as an internal standard. It is seen that abundant SIRT1 is present at PDL of 33, but is scantly produced at PDL 49.

### Example 2

### Expression and Suppression of SIRT1 Under Various Conditions

293T cells (human kidney cells; American Type Culture Collection (ATCC), Manassas, Va.) were transfected with the retroviral vector pBABE-puro (pBABE; Morgenstern, J P et al. Nucleic Acids Res. 1990; 18: 3587-3596) harboring various SIRT1 nucleic acid sequences, or empty control, using Fugene 6 transfection reagent (Roche Diagnostics Corp., Indianapolis, Ind.). They were simultaneously infected with the packaging plasmid containing a gag-pol expression plasmid (pVPack vector system, Stratagene, La Jolla, Calif.) and the VSV-G expression vector pUMVC3 (Stewart S A et al. RNA. 2003; 9(4): 493-501). The media containing progeny virus was collected and used to infect WI-38 cells for 3-6 hours in the presence of 8 ug/ml polybrene (Sigma Aldrich, St. Louis, Mo.). The medium was changed to a fresh MEM medium and the cells were incubated for an additional 48 hours. They were selected with puromycin (Sigma Aldrich) for 48 hours, and then trypsinized and were seeded at a concentration of 300,000 cells in 10 cm plates. The cells were harvested at PDL 39, and the various proteins were assayed by Western blotting. Anti-SIRT6 and anti-SIRT7 antibodies were rabbit polyclonal antibodies obtained from Dr. Ethan Ford, Dept. of Biology, Massachusetts Institute of Technology, Cambridge, Mass.

The results are shown in FIG. 2. In FIG. 2, C designates transfection with control pBABE-puro vector, Ti designates wild type SIRT1 (GenBank Acc. No. NP 036370), Ti HY designates the engineered SIRT1-HY mutant having a putatively defective deacetylation site described in Reference Example 2, T6 designates transfection with the coding sequence for murine SIRT6 (GenBank Acc. No. BC052763), and T7 designates transfection with the coding sequence for murine SIRT7 (GenBank Acc. No. BC026650). It is seen that at PDL 39 all four SIRT proteins, including the deletion mutant of SIRT1, are abundantly expressed in WI-38 cells.

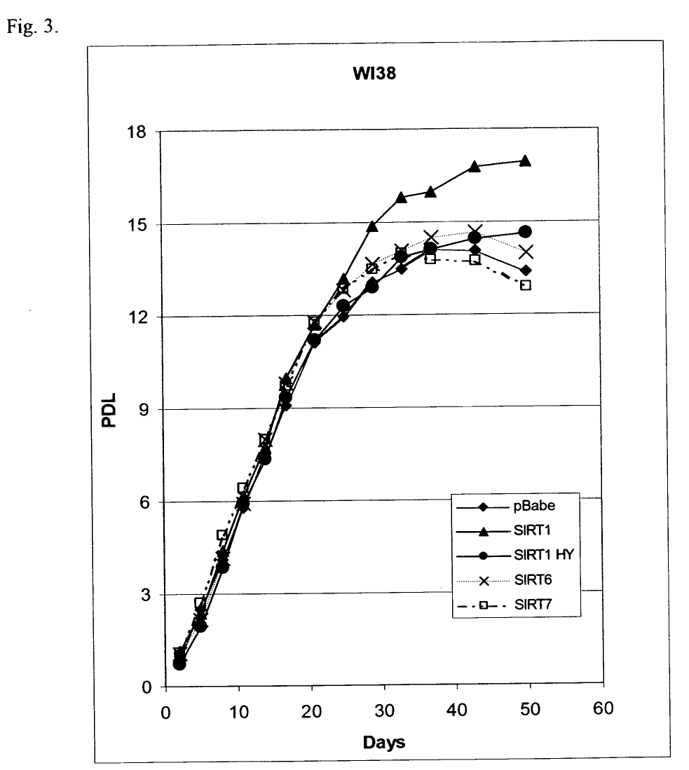

Using similarly prepared transfected cells and controls, the PDL was assessed as a function of time after seeding the transfected WI-38 cells on plates. The results are shown in FIG. 3. It is seen that at later times, the cells transfected with SIRT1 have undergone more doubling than other cells, including those transfected with the genes for SIRT6 and SIRT7, and the enzymatically inactive mutant of SIRT1. At senescence the SIRT1 cells have attained a higher PDL than the controls and the deletion mutant of SIRT1.

### Example 3

### beta-Galactosidase Activity in WI-38 Cells

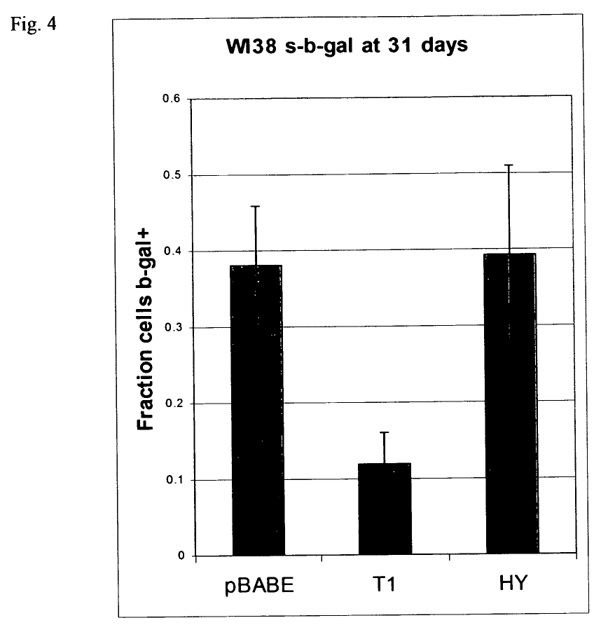

beta-Galactosidase activity is correlated with cell senescence and the attainment of the Hayflick limit (Dimri, G et al., PNAS 1995; 92: 9363-7). WI-38 cells were transfected as described in Example 2. The cultured cells were stained for beta-galactosidase activity as follows. Cells were fixed in 2% formaldehyde/0.2% glutaraldehyde. Fixed cells were incubated at 37° C. with fresh beta-galactosidase stain solution (sodium phosphate buffer (pH 6.0) containing 1 mg of X-Gal per ml/40 mM citric acid, 5 mM potassium ferrocyanide, 5 mM potassium ferricyanide, 150 mM NaCl, and 2 mM MgCl₂). Staining was detected by light microscopy following overnight incubation and the fraction of stained cells was assessed. The results are shown in FIG. 4. The designations T1 and HY are the same as in Example 2. It is seen that cells transfected with active SIRT1 (T1) have reduced beta-galactosidase activity compared to control. Transfection with enzymatically inactive SIRT1 (HY) shows the same degree of beta-galactosidase activity as the vector control (pBABE).

### Example 4

### SIRT1 Expression in MRC-5 Cells

MRC-5 cells (ATCC) are primary lung fibroblasts derived from a 14-week old human embryo. They constitutively express the RNA template component of telomerase (hTR). MRC-5 cells senesce after about 60 population doublings. Overexpression of hTERT (Franco S, Exp. Cell Res. 2001; 268: 14-25) extends the life span of MRC-5 cells.

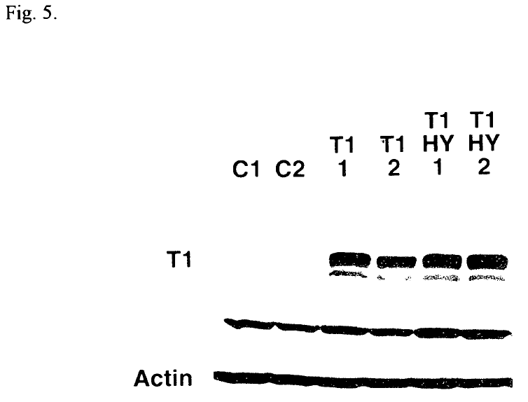

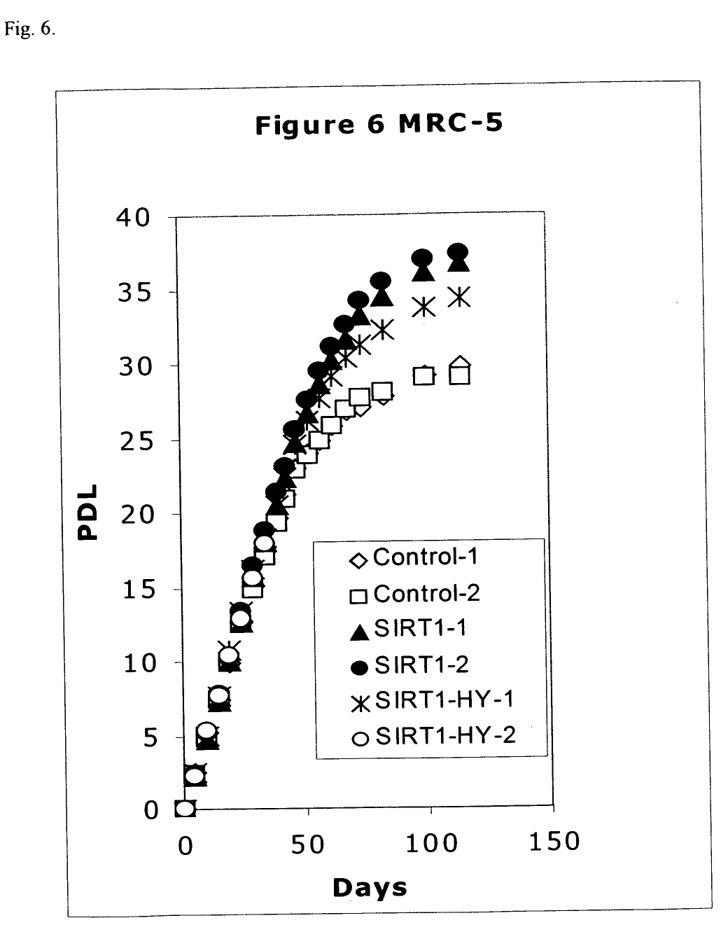

An experiment similar to that described in Example 2 was performed using MRC-5 cells. FIG. 5 shows a Western blot that demonstrates the expression of wild type SIRT1 and SIRT1-HY in MRC-5 upon transfection with the appropriate plasmids. Only wild type SIRT1, however, induces extension of PDL compared to empty vector controls (see FIG. 6). (In FIG. 6, the suffixes “−1” and “−2” represent separate replicate experiments. The experiment for SIRT1-HY-2 ended after 34 days.). The SIRT1-HY sample gives results that are intermediate between wild type SIRT1 and control. (A second SIRT1-HY sample did not attain completion of the experiment and is not shown.) It is concluded from the results of this Example that SIRT1 has the ability to extend PDL in the MRC-5 human cell line, in addition to showing the same property in WI-38 cells.

### Example 5

### Detection of Senescence in MRC-5 Cells Transfected with SIRT1

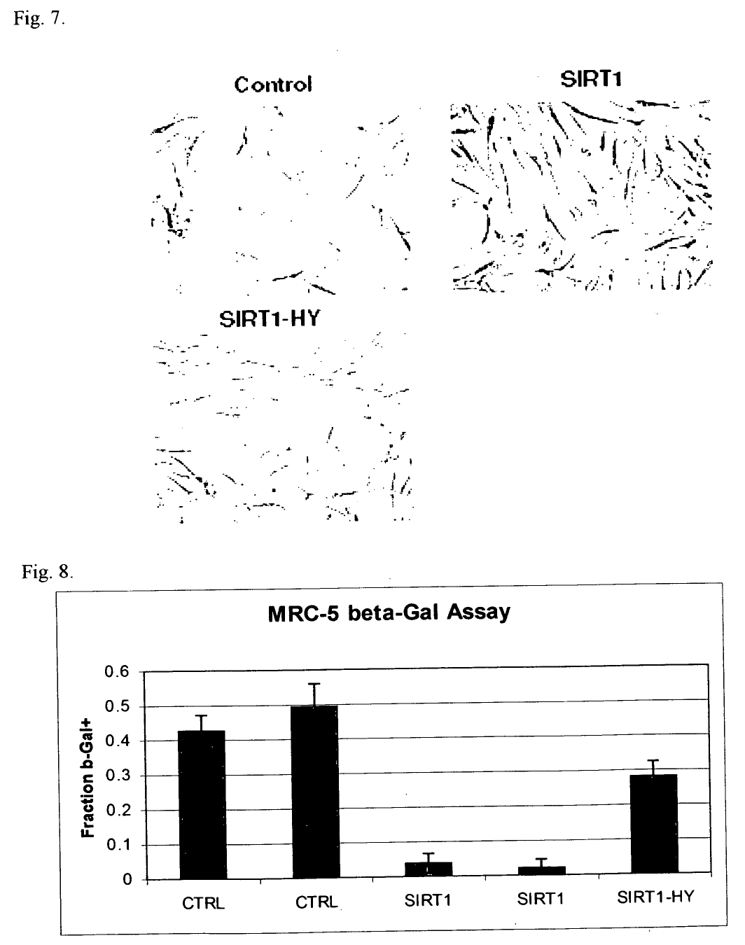

beta-Galactosidase activity was assessed in transfected MRC-5 cells (prepared as in Example 4) using the procedure described in Example 3. The cells were grown for 57 days and then stained with X-gal for beta-galactosidase activity. Photomicrographs of the results for three cases of transfected cells are shown in FIG. 7. The two panels on the left show results obtained with control cells, transfected with empty vector (top; PDL 24.78) and with a vector containing SIRT1-HY (bottom; PDL 27.69). Many cells in these panels contain large regions stained blue by X-gal. The panel on the right presents the results obtained with MRC-5 cells transfected with SIRT1 (PDL 29.48). No cells have the large areas of staining seen in the left-hand panels.

An evaluation of the fraction of cells from the three groups at day 57, stained with X-gal, is presented in FIG. 8. In FIG. 8, the PDL values for each sample are: for both CTRLs 24.78, for the SIRT1 samples 28.58 and 29.48, and for SIRT1-HY 27.69. It is seen that transfection with wild type SIRT1 reduces the number of cells with beta-galactosidase staining almost to zero. The cells transfected with SIRT1-HY show staining almost at the level of the negative control.

The results in this Example demonstrate inhibition of senescence-associated beta-galactosidase activity in a second human cell line, in addition to the similar finding with WI-38 cells described in Example 3.

1. An isolated polynucleotide comprising a nucleotide sequence chosen from the group consisting of:
a) a nucleotide sequence encoding a variant SIRT1 polypeptide whose amino acid sequence is at least 90% identical to an amino acid sequence that differs from the sequence given by SEQ ID NO:1 by one amino acid residue;
b) a nucleotide sequence complementary to a nucleotide sequence described in a);
c) a nucleotide sequence that is a fragment of any of the nucleotide sequences of a) or b); and
d) a nucleotide sequence that hybridizes to a nucleotide sequence given by a) through c).

2. The polynucleotide described in claim 1 wherein the variant polypeptide exhibits at least one biological activity of SIRT1.

3. An isolated variant SIRT1 polypeptide comprising a sequence chosen from the group consisting of:
a) a polypeptide whose amino acid sequence is at least 90% identical to an amino acid sequence that differs from the sequence given by SEQ ID NO:1 by one amino acid residue; and
b) an amino acid sequence that is a fragment of the amino acid sequence given in a).

4. The variant polypeptide described in claim 3 wherein the polypeptide exhibits at least one biological activity of SIRT1.

5. A method of extending the population doubling of a vertebrate cell comprising contacting the cell with a nucleic acid comprising a sequence described in claim 1, or with a sequence encoding the polypeptide of SEQ ID NO:1 or a fragment thereof.

6. The method described in claim 5 wherein the cell is a mammalian cell.

7. The method described in claim 5 wherein the cell is a human cell.

8. The method described in claim 5 wherein the cell is in vitro or ex vivo.

9. The method described in claim 5 wherein the cell is in vivo.

10. The method described in claim 5 wherein the cell is a cardiac myocyte, a neuron, a glial cell, a kidney cell, an endothelial cell, a myoblast, a muscle cell, an osteoblast, an osteoclast, a fibroblast, a keratinocyte, or a dermal, epidermal, or mucosal epithelial cell.

11. A method of inhibiting or delaying the expression in a vertebrate cell of a protein having biological activity associated with loss of population doubling in the cell, the method comprising contacting the cell with a nucleic acid comprising a sequence described in claim 1, or with a sequence encoding the polypeptide of SEQ ID NO:1 or a fragment thereof.

12. The method described in claim 11 wherein the cell is a mammalian cell.

13. The method described in claim 11 wherein the cell is a human cell.

14. The method described in claim 11 wherein the cell is in vitro or ex vivo.

15. The method described in claim 11 wherein the cell is in vivo.

16. The method described in claim 11 wherein the cell is a cardiac myocyte, a neuron, a glial cell, a kidney cell, an endothelial cell, a myoblast, a muscle cell, an osteoblast, an osteoclast, a fibroblast, a keratinocyte, or a dermal, epidermal, or mucosal epithelial cell.

17. The method described in claim 11 wherein the inhibiting or delaying is effective to inhibit or delay a differentiation process in the cell.

18. A method of treating a pathology, disease or medical condition in a subject, wherein the pathology, disease or medical condition responds to an SIRT1 polypeptide, the method comprising administering a nucleic acid comprising a sequence described in claim 1, or a sequence encoding the polypeptide of SEQ ID NO:1 or a fragment thereof, to the subject in an amount effective to attenuate or ameliorate the pathology.

19. The method described in claim 14 wherein the subject is a human.

20. The method described in claim 14 wherein the pathology, disease or medical condition is chosen from the group consisting of myocardial infarction, cerebrovascular stroke, a kidney disease, a neurological disease, a traumatic wound, a surgical wound, a fractured bone, a bone having a surgical wound, and a condition of a dermal, epidermal, or mucosal epithelial surface.

21. A vertebrate cell that contains a heterologous nucleic acid comprising a sequence described in claim 1, or a sequence encoding a fragment of the polypeptide of SEQ ID NO:1.

22. The vertebrate cell described in claim 17 wherein the population doubling of the cell is extended with respect to the population doubling of a cell not containing the heterologous nucleic acid.

23. The vertebrate cell described in claim 17 wherein the polypeptide possesses at least one biological function of wild type SIRT1.

24. The vertebrate cell described in claim 17 wherein the cell is in vitro or ex vivo.

25. The vertebrate cell described in claim 17 wherein the cell is in vivo.

26. The vertebrate cell described in claim 17 wherein the nucleic acid further comprises one or more of an enhancer sequence, a promoter sequence, and a polyadenylation sequence each of which is operably linked to the SIRT1 sequence.

27. The vertebrate cell described in claim 17 wherein the expression in the vertebrate cell of a protein having biological activity associated with loss of population doubling in the cell is inhibited or delayed.

28. The vertebrate cell described in claim 17 wherein a differentiation process in the cell is inhibited or delayed.

29. The vertebrate cell described in claim 17 wherein the cell is a cardiac myocyte, a neuron, a glial cell, a kidney cell, an endothelial cell, a myoblast, a muscle cell, an osteoblast, an osteoclast, a fibroblast, a keratinocyte, or a dermal, epidermal, or mucosal epithelial cell.

30. A transgenic mammal a majority of whose cells harbor a transgene comprising a nucleic acid sequence described in claim 1 or a sequence encoding the polypeptide of SEQ ID NO:1 or a fragment thereof.

31. The transgenic mammal described in claim 26 wherein the number of the transgenes in the majority of the cells is higher than the number of SIRT1 sequences in the cells of a nontransgenic mammal of the same species.

32. The mammal described in claim 26 wherein the transgene further comprises a promoter operably linked to the sequence.

33. The mammal described in claim 26 wherein the life span of the mammal is increased with respect to a nontransgenic mammal of the same species.

34. An antibody that binds immunospecifically to a polypeptide described in claim 3.

35. A method of determining whether the amount of an SIRT1 polypeptide in a sample differs from the amount of the SIRT1 polypeptide in a reference, wherein the method comprises the steps of:
a) providing a sample suspected to include the SIRT1 polypeptide;
b) contacting the sample with a specific binding agent that binds an SIRT1 polypeptide under conditions that assure binding of the SIRT1 polypeptide to the specific binding agent; and
c) determining whether the amount of the specific binding agent that binds to the sample differs from the amount of the specific binding agent that binds to a reference under the same conditions used in step b), wherein the reference comprises a standard or reference amount of the SIRT1 polypeptide.

36. The method described in claim 22 wherein the specific binding agent is an antibody.

37. A method of contributing to the diagnosis or prognosis of, or to developing a therapeutic strategy for, a disease or pathology in a first subject, wherein the disease or pathology responds to treatment with an SIRT1 polypeptide and wherein the amount of SIRT1 polypeptide in the pathology is known to differ from the amount of the SIRT1 polypeptide in a nonpathological state, the method comprising the steps of:
providing a sample from the first subject suspected to include the SIRT1 polypeptide;
contacting the sample with a specific binding agent that binds an SIRT1 polypeptide under conditions that assure binding of the SIRT1 polypeptide to the specific binding agent; and
determining whether the amount of the specific binding agent that binds to the sample differs from the amount of the specific binding agent that binds to a reference under the same conditions used in step b), wherein the reference is provided from a second subject known not to have the pathology;
thus contributing to the diagnosis or prognosis of, or to developing a therapeutic strategy for, the pathology.

38. The method described in claim 22 wherein the specific binding agent is an antibody.


SEQUENCE LISTING (5 sequences)

SEQ ID NO: 1 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 2 - DNA, 4107 bp, Homo sapiens
  CDS (54)...(2297)
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 3 - DNA, 33 bp, Homo sapiens
ggatccaccatggcggacgaggcggccctcgcc

SEQ ID NO: 4 - DNA, 31 bp, Homo sapiens
gtctagagtggaacaattcctgtacctgcac

SEQ ID NO: 5 - DNA, 29 bp, Homo sapiens
gaggaggagatgcgcagttccggccgccc